(19) United States
(12) Patent Application Publication
(10) Pub. No.: US 2009/0075941 A1
(43) Pub. Date: Mar. 19, 2009

(54) TREATING NEOPLASMS

(75) Inventors: Katri Selander, Birmingham, AL (US); Pierre Triozzi, Shaker Heights, OH (US)

Correspondence Address: FISH & RICHARDSON P.C., P.O BOX 1022, Minneapolis, MN, 55440-1022, US

(73) Assignee: THE UAB RESEARCH FOUNDATION, Birmingham, AL (US)

(21) Appl. No.: 12/297,983

(22) PCT Filed: Apr. 11, 2007
(86) PCT No.: PCT/US2007/066381

§ 371 Date: Nov. 12, 2008

Related U.S. Application Data
(60) Provisional application No. 60/745,339, filed on Apr. 21, 2006.

(51) Int. Cl. A61K 31/663; A61P 35/00

(52) U.S. Cl. 514/89; 514/108; 514/94

(57) ABSTRACT

Methods of treating a subject with neoplasm (e.g., mesothelioma) or at risk of developing neoplasm by administering a mevalonate pathway inhibitor such as a nitrogen-containing bisphosphonate are disclosed. Examples of nitrogen-containing bisphosphonates include alendronate, ibandronate, minodronate, neridronate, olpadronate, pamidronate, risedronate, and zoledronate. The methods can further include the administration of a p38 inhibitor. Further disclosed are compositions and kits including a nitrogen-containing bisphosphonate and optionally a p38 inhibitor.

### CROSS-REFERENCE TO RELATED APPLICATIONS

This application claims the benefit of U.S. Provisional Application Ser. No. 60/745,339, filed on Apr. 21, 2006, which is incorporated herein by reference in its entirety.

### BACKGROUND

Mesothelioma, an asbestos-related neoplasm of the pleural and peritoneal space, occurs in approximately 10,000 patients yearly worldwide. Due to the long latency period for tumor development and the widespread use of asbestos for many years, the incidence is expected to rise until the year 2020. Thus, it is estimated that mesothelioma deaths will double over the next 20 years. The biological behavior is distinct from other solid tumors in that mesothelioma tends to grow in a sheet-like fashion, covering the surface of pleura or peritoneum. It shows little tendency to invade, especially early in the course of the disease. Mesothelioma typically recurs even after the most aggressive attempts at surgical resection and is poorly responsive to radiotherapy and chemotherapy. The survival of patients with mesothelioma ranges between 4 and 12 months. New treatment modalities are needed.

### SUMMARY

Provided herein are methods of treating or preventing neoplasms, like mesotheliomas. The methods reduce the proliferation of mesothelioma cells and tumors and prolong survival of subjects with mesotheliomas or at risk for mesotheliomas. The methods include administration of a mevalonate pathway inhibitor and/or bisphosphonate to a subject in need of treatment for mesothelioma or at risk for developing mesothelioma. Mevalonate pathway inhibitors include bisphosphonates (BPs), such as nitrogen-containing bisphosphonates. Examples of nitrogen-containing bisphosphonates include alendronate, ibandronate, minodronate, neridronate, olpadronate, pamidronate, risedronate, and zoledronate. The methods can further include the administration of a p38 inhibitor. Also disclosed are compositions including a nitrogen-containing bisphosphonate and a p38 inhibitor. Further disclosed are kits containing a composition comprising a nitrogen-containing bisphosphonate and instructions for administering the composition to a subject with mesothelioma or at risk of developing mesothelioma.

The details of one or more embodiments are set forth in the accompanying drawings and the description below. Other features, objects, and advantages of the methods and compositions will be apparent from tire description and drawings, and from the claims.

### DESCRIPTION OF DRAWINGS

FIGS. 1(a-b) show that nitrogen-containing BPs (N-BPs) induce the accumulation of unprenylated Rap1A in mesothelioma cells. Accumulation of unprenylated Rap1A was detected in (a) AB12 and in (b) AC29 cells after treatment for 24 h with the indicated concentrations of risedronate or zoledronate. Addition of 25 μM geranylgeraniol (GG), which is the end product of the mevalonate pathway that N-BPs inhibit, reversed the accumulation of risedronate and zoledronate-induced impaired prenylation of Rap1A. The same volume of ethanol (as a vehicle control) did not reverse the impaired prenylation of Rap1A. This was shown by lysing the treated cells and then running the samples in Western blot analysis. The levels of unprenylated Rap1A (upper panel) were used as a surrogate marker to detect the inhibition of the mevalonate pathway, using the antibody SC-1482. The blots, which represent replicate experiments, were stripped and total Rap1 was detected with the antibody SC-65, to show that the effects were not due to a loading error.

FIGS. 2(a-b) show that geranylgeraniol reverses the nitrogen-containing BP-induced growth inhibition, (a) AB12 and (b) AC29 were cultured in the presence of indicated concentrations of risedronate or zoledronate, with 25 μM geranylgeraniol (GG) or the same volume of ethanol as a vehicle control. DNA-synthesis as an indicator of cell proliferation rate was measured with BrdU-incorporation after 5 days of treatment. Data are expressed as % of PBS-control and represent mean±S.D., n=5. ** p<0.01, *** p<0.001 vs. the corresponding vehicle control.

FIGS. 3(a-b) show that nitrogen-containing BPs induce p38 phosphorylation. AB12 and AC29 cells were cultured for 24 h in the presence of the indicated concentrations of risedronate, zoledronate or PBS, with 25 μM geranylgeraniol (GG) or ethanol as a vehicle control. Phosphorylation of p38 was detected in (a) AB12 and in (b) AC29 cell lysates in Western blots, using phospho-p38 (upper panel) and after stripping of the same blot, total p38 (lower panel) specific antibodies

FIGS. 4(a-b) show that inhibition of p38 augments n-BP induced growth inhibition. AB12 and AC29 cells were cultured with the indicated concentrations of (a) risedronate or (b) zoledronate, with the specific p38 inhibitor SB202190 (10⁻⁵ M) or the same volume of an inactive control compound SB202474, DNA-synthesis as an indicator of cell proliferation rate was measured with BrdU-incorporation after 5 days of treatment. Data are expressed as % of PBS-control and represent mean±S.D., n=5. *** p<0.001 vs. the corresponding vehicle control.

FIGS. 5(a-c) show that risedronate and zoledronate mediate antitumor activity in vivo. (a) AB12 cells were inoculated subcutaneously into the flanks of mice. Ten days later, groups of 10 mice were treated subcutaneously with PBS, zoledronate (0.5 mg/kg) or risedronate (15 mg/kg) every six days for a total of four injections. Data are expressed as tumor volume±SE. (b) AB12 cells were inoculated into the peritoneal cavities of mice. Six days later, groups of 12 mice were treated by intraperitoneal injection of zoledronate (0.5 mg/kg), risedronate (15 mg/kg) or an equal volume of PBS three times a week for two weeks. Data are expressed as % of survival. (c) AC29 cells were inoculated into the peritoneal cavities of mice (n=10). Six days later, the mice were treated by intraperitoneal injection of zoledronate (0.5 mg/kg) or with an equal volume of PBS three times a week for two weeks. Data are expressed as % of survival.

FIGS. 6(a-c) show that pyrophosphate-resembling bisphosphonates prevent the nitrogen-containing bisphosphonate-induced accumulation of unprenylated Rap1A in breast cancer and mesothelioma cells. (a) AB-12 and (b) MDA-MB-231 cells were treated for 24 h with PBS as a vehicle control or with the indicated concentrations of the various bisphosphonates, alone or in combination. Expression of unprenylated Rap1A (u-Rap1A, upper panels) and after stripping and re-blotting, total Rap1 (lower panels) was detected in Western blots, using antibodies that detect different forms of the protein, (e) MDA-MB-231 cells were also treated with 10 ng/ml of LPS, IL-1β, TNF-α alone or with 10⁻⁴ M alendronate in combination with the indicated cytokines or LPS, clodronate (clo, 10⁻³ M) or geranylgeraniol (GG, 25 μM) for 24 h.

FIGS. 7(a-b) show that pyrophosphate-resembling bisphosphonates prevent the nitrogen-containing bisphosphonate-induced phosphorylation of p38 in cancer cells. (a) AB-12 and (b) MDA-MB-231 breast cancer cells were treated for 24 h with PBS as a vehicle control or with the indicated concentrations of the various bisphosphonates, alone or in combination with clodronate (clo, 10⁻³ M) or etidronate (eti, 10⁻³ M) for 24 h. The phosphorylation status of p38 was studied in Western blots, using phospho-p38 (upper panels) and total p38 (lower panels) specific antibodies.

FIGS. 8(a-d) show that pyrophosphate-resembling bisphosphonates prevent the growth inhibitory effects of nitrogen-containing bisphosphonates in cancer cells, (a) The indicated cells were treated with PBS or 10⁻³ M clodronate or etidronate, with or without vehicle, 1 mM CaCl₂ or 1 mM EGTA for 72 h and viability was measured with MTS-assays. Data are expressed as % of PBS-control in the corresponding groups. Mean±S.D., n=10-15. * P<0.05, ** P<0.01, *** P<0.001 vs. vehicle-treated group, (b) AB-12, (c) MDA-MB-231 or d) J774 cells were treated with PBS or with the indicated nitrogen-containing bisphosphonates (zoledronate, risedronate, alendronate), in combination with vehicle, 1 mM CaCl₂, 1 mM EGTA, 1 mM EGTA+1 mM CaCl₂, or with 10⁻³ M pyrophosphate-resembling bisphosphonates (clodronate or etidronate), with or without 1 mM CaCl₂. Cell viability was measured 72 h later with MTS-assays. Data are expressed as % of corresponding PBS-control for each treatment, mean±S.D., n=15-20, * P<0.05, ** P<0.01, *** P<0.001 vs. corresponding vehicle-treatment group. # P<0.05, ## P<0.01, ### P<0.001 vs. corresponding treatment containing Ca²⁺.

FIGS. 9(a-d) show that MDA-MB-231 cells express connexin-43 but not γλ-TCR. (a) Connexin-43 expression was detected on MDA-MB-231 cell membranes by immunofluorescence. (b) Western blots showed the effects of the indicated bisphosphonates on the expression of connexin-43 in MDA-MB-231 cells after 24 h. The same blots were stripped and reblotted for actin, to show equal loading. Flow cytometry analysis of γλ-T-cell receptor was used to monitor (e) MDA-MB-231 and (d) cultured human mononuclear cells as a positive staining control. PE-conjugated anti-γλ-TCR mAb data is shown in (c) an (d) with solid black lines indicating count level and total count level of PB-conjugated isotypic control mAb is shown with a short line at the peak count value.

FIGS. 10(a-b) show that mesothelioma tumors exhibit higher Tc99m-medronate uptake than breast cancer tumors, (a) Accumulation of Tc99m-medronate was detected in bones, as well as in the subcutaneous tumors in both AB-12 and MDA-MB-231 bearing mice. The images represent CT- (left panel) and SPECT-(right panel) images of an AB-12 tumor bearing mouse. (b) The % dose retention in the indicated target tissues of MDA-MB-231 or AB-12 tumor bearing mice. Mean±S.D., n=10-15 indicating the number of tissues analyzed. * P< 0.05 vs. the MDA-MB-231 tumor.

FIGS. 11 (a-b) show that mesothelioma and breast tumors exhibit calcification. a) The results of H&E (left panels) and Von Kossa stainings (right panels) of AB-12 and MDA-MB-231 tumors as shown. Intracellular staining is seen in AB-12 cells in areas of necrosis. In tumors formed by MDA-MB-231 cells, also cells within the vicinity of necrotic cells exhibit positive staining. b) For comparison, viable tumor is shown, with positive staining in only 2 cells (arrow).

### DETAILED DESCRIPTION

Provided herein are methods of treating or preventing neoplasms, like mesotheliomas, by administering to subjects in need thereof a therapeutic dose of a compound or composition that inhibits the mevalonate pathway. An example of a mevalonate pathway inhibitor is a nitrogen-containing bisphosphonate.

Bisphosphonates (BPs) are synthetic analogs of the naturally occurring pyrophosphate. Depending on their molecular structure these drags can be divided into pyrophosphate-resembling (p-BPs, such as clodronate) and nitrogen-containing BPs (n-BPs, such as alendronate, pamidronate, risedronate and zoledronate). At the cellular level the different BPs have different mechanisms of action; n-BPs inhibit the mevalonate pathway, whereas the effects of p-BPs lire mediated via infra-cellular ATP-like analogs. The main effect of all BPs is their ability to inhibit osteoclast-mediated bone resorption. These drugs are therefore widely clinically used in the treatment of metabolic bone diseases that are due to increased bone resorption, such as osteoporosis. Nitrogen-containing bisphosphonates, for example, act on bone metabolism by binding and blocking the enzyme farnesyl diphosphate synthase (FPPS) in the HMG-CoA reductase pathway (mevalonate pathway).

BPs also inhibit the osteolytic complications of bone metastases of solid tumors and multiple myeloma. Data from animal models suggest that, in addition to osteoclast inhibition at the site of bone metastasis, these drugs may also inhibit cancer cell proliferation in bone. Especially the newer n-BPs have also been suggested to actually inhibit the cancer spread to bones in animal models. Although these drugs inhibit significantly the growth of various cancer cells in vitro, they have not previously proven to be acceptable agents in preventing or treating tumor growth at visceral sites in various animal models of cancer.

Generally, bisphosphonates have a P—C—P backbone as shown in structure I:

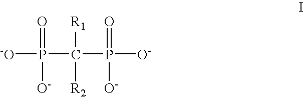

R₁ is typically referred to as the short side chain. R₁ can be, for example, —H, —Cl, or —OH. R₂ is typically called the long side chain. The R₂ sidechain contains a nitrogen in nitrogen-containing bisphosphonates. R₂ in nitrogen-containing bisphosphonates can be, for example, —CH₂—CH₂—NH₂; —(CH₂)₅—NH₂; —(CH₂)₂N(CH₃)₂; —(CH₂)₃—NH₂. Further examples of possible R₂ side chains in nitrogen-containing bisphosphonates include structures II. III, IV, and V:

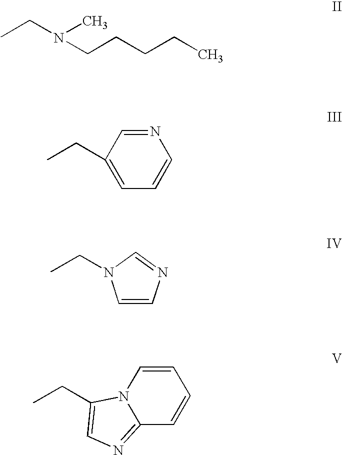

Nitrogen containing bisphosphonates useful in the compositions and methods described herein include, for example, alendronate (R₁=—OH; R₂=—(CH₂)₃—NH₂), ibandronate (R₁=—OH; R₂=Structure II), minodronate (R₁=—OH; R₂=Structure V), neridronate (R₁=—OH: R₂=—(CH₂)₅—NH₂), olpadronate (R₁=—OH; R₂=—(CH₂)₂N(CH₃)₂), pamidronate (R₁=—OH; R₂=—CH₂—CH₂—NH₂), risedronate (R₁=—OH; R₂=Structure III), and zoledronate (R₁=—OH; R₂=Structure IV).

In the methods provided herein, the mevalonate pathway inhibitor is optionally administered in combination with other therapeutic modalities or treatments. For example, the mevalonate pathway inhibitor optionally is administered in combination with a p38 inhibitor (e.g. SB202190). By in combination is meant that the mevalonate pathway inhibitor is administered prior to, simultaneously with, or after the p38 inhibitor. When administered simultaneously, the mevalonate pathway inhibitor and the p38 inhibitor may be provided at the same time in different compositions or may be administered in the same composition. Thus, provided herein is a composition comprising a nitrogen-containing bisphosphonate and a p38 inhibitor.

Prior to treatment with a mevalonate pathway inhibitor, the subject may be first identified as having mesothelioma or may be first identified as being at risk for developing mesothelioma. Mesothelioma is used throughout as an example of neoplasm. The methods and compositions described herein are useful in treating other neoplasms as well. Identification of mesothelioma in a subject includes diagnostic methods presently used in the art or methods to be developed. Identification of subjects at risk for mesothelioma may be based on a known exposure of the subject to asbestos or because of early clinical or preclinical symptoms.

People at risk of developing mesothelioma later in their life include those exposed to asbestos. Millions of people worldwide have been exposed to asbestos and, therefore, the incidence of this disease is quickly increasing. The most common presenting features in patients with peritoneal (abdominal) malignant mesothelioma are distention due to ascites, abdominal pain and occasionally organ impairment, such as bowel obstruction. The most common sign of malignant mesothelioma in the chest is pleural effusion and shortness of breath. It has been recently shown that serum osteopontin levels can be used to distinguish people with exposure to asbestos who do not have mesothelioma from those that were exposed to asbestos and who have pleural mesothelioma. In addition to pleura and peritoneum, malignant mesothelioma can occur on any serous surface of the body, including pericardium and tunica vaginalis and symptoms from these organs also can be present.

Mesothelioma can be diagnosed with imaging studies (X-rays to show pleural effusion in the chest or bowel distention in the abdominal cavity). Additional diagnostic imaging methods include MRI, ultrasound and PET scans, in addition to serum osteopontin levels, serum mesothelin-related protein (SMRP) measurements are used in diagnosis and treatment follow-up. Cytologic analysis is done from pleural or ascitic fluid or from the tumor by fine-needle biopsies, to confirm the presence of malignant mesothelioma cells. Histopathological analysis from a tumor biopsy is also often needed to confirm the diagnosis. Thus, identification of a person with mesothelioma or at risk for mesothelioma can be determined with a number of different methods.

Also provided herein is a kit containing a composition comprising a nitrogen-containing bisphosphonate and a p38 inhibitor and instructions for administering the composition to a subject with mesothelioma or at risk of developing mesothelioma.

The compositions described herein may be administered orally, parenterally (e.g., intravenously), by intramuscular injection, by intraperitoneal injection, transdermally, extracorporeally, topically or the like.

The compositions may be in solution or suspension. The compositions can be administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington's Science and Practice of Pharmacy, 21st Edition, ed. University of the Sciences in Philadelphia, Lippincott, Williams & Wilkins, Philadelphia Pa., 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.0 to about 8.2, Further carriers include sustained release preparations such as semipermeable matrices of solid hydrophobic polymers, e.g., films, liposomes or microparticles. It will be apparent to those persons skilled in the art that certain earners may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The terms therapeutic dose, effective amount, and effective dosage, are used interchangeably herein. The terms refer to the amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like. Generally, the dosage will vary with tire species, age, weight, condition, sex, and the type, extent, and severity of the disease in the patient, specific active agent used, route of administration, or whether other drugs are included in the regimen, and can be determined by one of skill in the art. Thus, it is not possible to specify an exact amount for every composition. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

Dosages for alendronate as used in the methods herein, for example, include for an average adult human about 0.1 mg to about 70 mg daily, and more particularly up to about 70 mg daily or up to about 40 mg/day, The dosages of alendronate alternatively expressed in mg/kg include, for example, alendronate in the range of about 0.05 mg/kg to about 1 mg/kg. The alendronate treatment may be continuous for a period of days or may be intermittent. For example, alendronate may be administered daily up to 6 months and preferably for about 2 months. For further example, alendronate may be administered once weekly for up to several years. Treatment can be reinitiated at the end of a treatment period as necessary.

Dosages for pamidronate as used in the methods herein, for example, include for an average adult human about 0.1 mg to about 120 mg daily, and more particularly up to about 90 mg daily, up to about 60 mg daily, or up to about 30 mg daily. The dosages of pamidronate alternatively expressed in mg/kg include, for example, pamidronate in the range of about 0.05 mg/kg to about 1.7 mg/kg. The pamidronate treatment may be continuous for a period of days or may be intermittent. For example, pamidronate may be administered daily, weekly, or monthly for up to 6 months or longer and preferably for about 2 months. Treatment could be reinitiated at the end of a treatment period as necessary.

Dosages for risedronate as used in the methods herein, for example, include for an average adult human about 0.1 mg to about 50 mg daily, and more particularly up to about 30 mg daily. The dosages of risedronate alternatively expressed in mg/kg include, for example, risedronate in the range of about 0.05 mg/kg to about 0.7 mg/kg. The risedronate treatment may be continuous for a period of days or may be intermittent. For example, risedronate may be administered daily up to 6 months and preferably for about 2 months. Treatment could be reinitiated at the end of a treatment period as necessary.

Dosages for zoledronate as used in the methods herein, for example, include for an average adult human about 0.1 mg to about 5 mg per dose, and more particularly up to about 4 mg per dose, which may be repeated every 3 to 4 weeks. The dosages of zoledronate alternatively expressed in mg/kg include, for example, zoledronate in the range of about 0.01 mg/kg to about 0.06 mg/kg. The zoledronate treatment may be continuous for a period of days or may be intermittent. Treatment could be reinitiated at the end of a treatment period as necessary.

As used throughout, by a subject is meant an individual. The term subject can include a mammal such as a primate or a human. The term subject can also include domesticated animals, such as cats, dogs, etc., livestock (e.g., cattle, horses, pigs, sheep, goats, etc.), laboratory animals (e.g., mouse, rabbit, rat, guinea pig, etc.) and birds.

A number of embodiments have been described. Nevertheless, it will be understood that various modifications may be made. For example, instead of a nitrogen-containing BP, other inhibitors of the mevalonate pathway are useful herein. Accordingly, other embodiments are within the scope of the claims.

As used in the specification and the appended claims, the singular forms a, an and the include plural referents unless the context clearly dictates otherwise. Thus, for example, reference to a small molecule includes mixtures of one or more small molecules, and the like.

Ranges may be expressed herein as from about one particular value, and/or to about another particular value. When such a range is expressed, another embodiment includes from the one particular value and/or to the other particular value. Similarly, when values are expressed as approximations, by use of the antecedent about, it will be understood that the particular value forms another embodiment. It will be former understood that the endpoints of each of the ranges are significant both in relation to the other endpoint, and independently of the other endpoint.

The examples below are intended to further illustrate certain embodiments, and are not intended to limit the scope of the claims.

### EXAMPLES

### Example 1

### Materials and Methods

Bisphosphonates. Risedronate was dissolved in phosphate buffered saline (PBS) and pH of the stock solution was set to 7.4 with NaOH. Zoledronate was diluted into cell culture medium. For animal studies both BPs were diluted into sterile 0.9% saline.

Cell Culture. The mouse mesothelioma cell lines AB12 and AC29, which have been well characterized models of mesothelioma, were used. AB12 and AC29 cells were cultured and maintained in complete medium consisting of high glucose DMEM (Mediatech, Washington, D.C.) supplemented with 10% heat-inactivated fetal calf serum (FCS), 100 units/ml penicillin, 100 μg/ml streptomycin, and 2 mM glutamine (Sigma, St. Louis, Mo.). All cell cultures were done in incubators in a 37° C. atmosphere of 5% CO2/95% air.

In vitro growth assay. Mesothelioma cells were plated in 96-well plates in normal culture medium and treated for the indicated periods of time with various concentrations of zoledronate, risedronate or PBS with or without the p38 inhibitor SB202190 or the inactive control compound SB207420 (Calbiochem, both at the final concentration of 10−5 M), 25 μM geranylgeraniol (cold, all trans, American Radiolabeled Chemicals, St. Louis, Mo.) or the same volume of ethanol as a vehicle control, DNA-synthesis was measured as an indication of cell proliferation, using non-isotopic bromodeoxyuridine (BrdU) incorporation immunoassays (Exalpha Biologicals, Watertown, Mass.), according to the manufacturer's instructions. Briefly, 103 cells were plated onto 96-well plates in 100 μl of normal culture medium. The cells were then treated with the indicated agents for various times. BrdU was added to the wells for the final 24 h and incorporated BrdU was detected with sequential additions of monoclonal mouse anti-BrdU antibody and HRP-conjugated anti-mouse antibody. After addition of the substrate for HRP, intensity of the colored reaction product, which is proportional to the amount of BrdU incorporated into the cells, was read with spectrophotometer at 450 nM.

Western blotting. AB12 and AC29 cells were plated on 6-well plates in normal culture medium until near confluency. The cells were then rinsed with sterile PBS and cultured for further 24 h in serum-free culture medium, in the presence or absence of 2×10⁻⁴-10⁻⁵ M risedronate, zoledronate or PBS control, with or without 25 μM geranylgeraniol, or the same volume of ethanol as a vehicle control. Culture medium was discarded and the cells were harvested in lysis buffer (20 mM Tris pH 7.4, 150 mM NaCl, 1 mM EDTA, 1 mM EGTA, 1% Triton, 2.5 mM sodium pyrophosphate, 1 mM β-glycerolphosphate, 1 mM Na₃VO₄, 1 μg/ml leupeptin (Cell Signaling Technology, Inc.; Danvers, Mass.)) and clarified by centrifugation. After boiling the supernatants in reducing SDS sample buffer, equal amounts of protein (˜50 μg) were loaded per lane and the samples were electrophoresed on 10% polyacrylamide SDS gel and transferred to a nitrocellulose membrane. Unprenylated Rap1A was detected with the antibody SC-1482 and total Rap1 (both prenylated and unprenylated forms of both Rap1A and Rap1B) was detected with the antibody SC-65 (Santa Cruz Biotechnology, Inc.; Santa Cruz, Calif.), according to the manufacturer's recommendations. The phosphorylation status of p38 was studied with anti-phospho-p38 and anti-total p38 antibodies (Cell Signaling Technology, Inc.), as recommended by the manufacturer. The protein bands were visualized by chemiluminescence using SuperSignal West Pico ECL kit (Pierce; Rockford, Ill.).

In vivo mesothelioma models. Female BALB/c mice, four to eight weeks of age, were obtained from the National Cancer Institute-Frederick Cancer Research Facility (Frederick, Md.) and were housed in the Pathogen-Free Rodent Shared Facility (Comprehensive Cancer Center, University of Alabama at Birmingham). All animal procedures were performed, in accordance with recommendations for the proper care and use of laboratory animals and were approved by the local IACUC. Subcutaneous (s.c.) and intraperitoneal (i.p.) mouse mesothelioma models were evaluated. In the s.c. model, 3×10⁶ AB12 cells were first injected s.c. into cohorts of BALB/c mice. Treatments with i.p. BPs or vehicle were started when tumors became palpable on day 10 and continued every six days for a total of 4 treatments. Tumor size was measured bidimensionally with calipers every two to three days and tumor volume calculated by the formula (length×width²)÷2. Mice were euthanized before tumors reached the size of 2000 mm³. In the i.p. model, AC29 or AB12 cells (5×10⁵/0.5 ml) were injected i.p, into cohorts of 10-12 BALB/c using a 26-gauge needle. Treatment was initiated 6 days after tumor inoculation and the mice were followed for survival. In the i.p. model risedronate (15 mg/kg), zoledronate (0.5 mg/kg) or PBS were administered i.p. three times a week for two weeks.

Statistical analysis. Kaplan-Meier survival curves were analyzed with the Mantel-Cox Log-rank test. Fisher exact test was used to examine differences in the proportion of tumors responding and proportion of mice surviving. Student's t test (two-tailed) was used to examine differences in growth assays and for the time to death/sacrifice. Results are expressed as mean±S.D. P< 0.05 was considered to be statistically significant.

Results

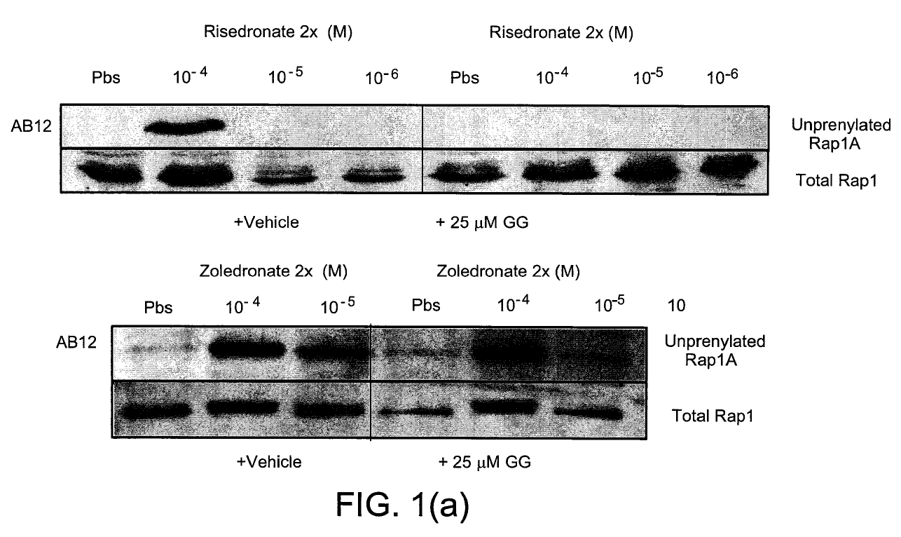

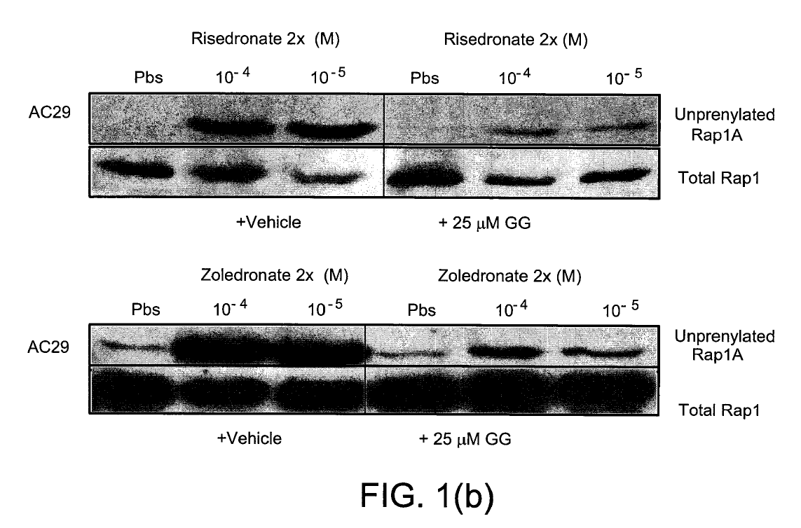

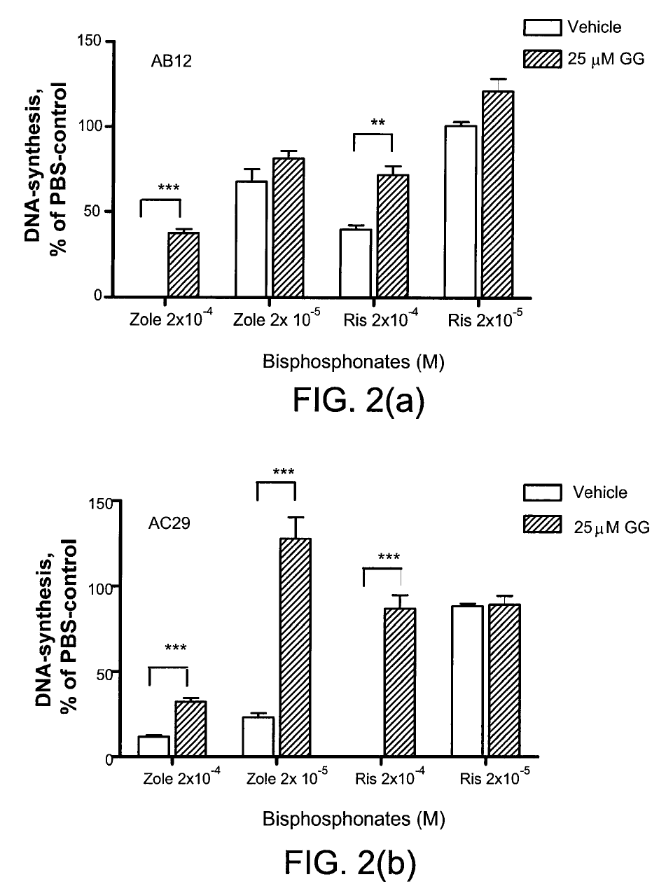

Risedronate and zoledronate effects on Rap1A accumulation and growth inhibition were partially reversed by geranylgeraniol in mesothelioma cells. Nitrogen-containing BPs have been previously shown to inhibit the growth of various epithelial cancer cells in vitro, via inhibiting the mevalonate pathway. This inhibition results in the depletion of intracellular prenyl-groups, such as geranylgeraniol, which are needed for the post-translational modification and activation of small GTP-binding proteins, such as Ras, Rho, Rac and Rap. For example, treatment with n-BPs has been shown to result in the accumulation of unprenylated Rap1A in CaCo-2 and leukemia cells. To investigate whether risedronate and zoledronate similarly inhibit the mevalonate pathway in mesothelioma cells, AB12 and AC29 cells were treated for 24 h with PBS or with 2×10⁻⁴-2×10⁻⁶ M risedronate or zoledronate, with 25 μM geranylgeraniol or the same volume of ethanol as a vehicle control. The cells were then lysed and prepared for Western blot analysis. Accumulation of unprenylated Rap1A was used as a surrogate marker for the inhibition of the mevalonate pathway. Zoledronate and risedronate induced a dose-dependent accumulation of unprenylated Rap1A in both cell lines, Risedronate-induced accumulation of unprenylated Rap1A was almost completely reversed by 25 μM geranylgeraniol in both cells. Zoledronate-induced accumulation of unprenylated Rap1A was partially reversed in both cells. Stripping and reblotting the membranes with the anti-total Rap1 antibody clearly indicated that the findings were not due to uneven loading of the gels (FIG. 1). Higher concentrations of geranylgeraniol were also tested and found effective, but since they compromised cell viability, they were not routinely used. Geranylgeraniol also reversed the BP-induced inhibition of DNA-synthesis in both cells, but the extent of this reversal was dependent on the cell line and the BPs used (FIG. 2).

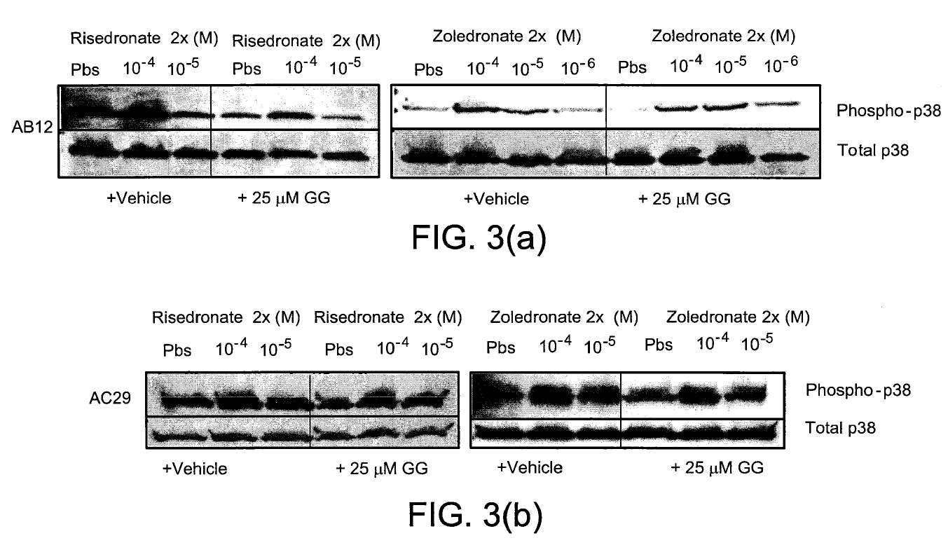

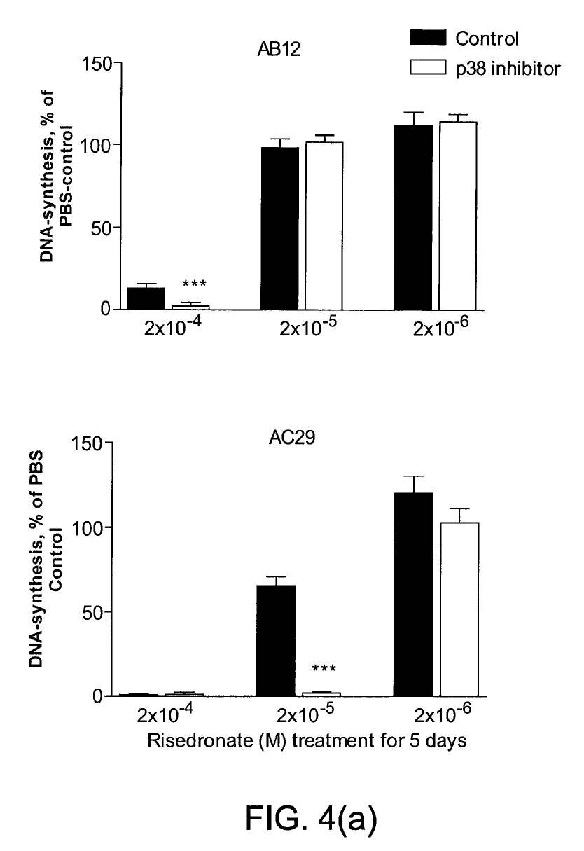

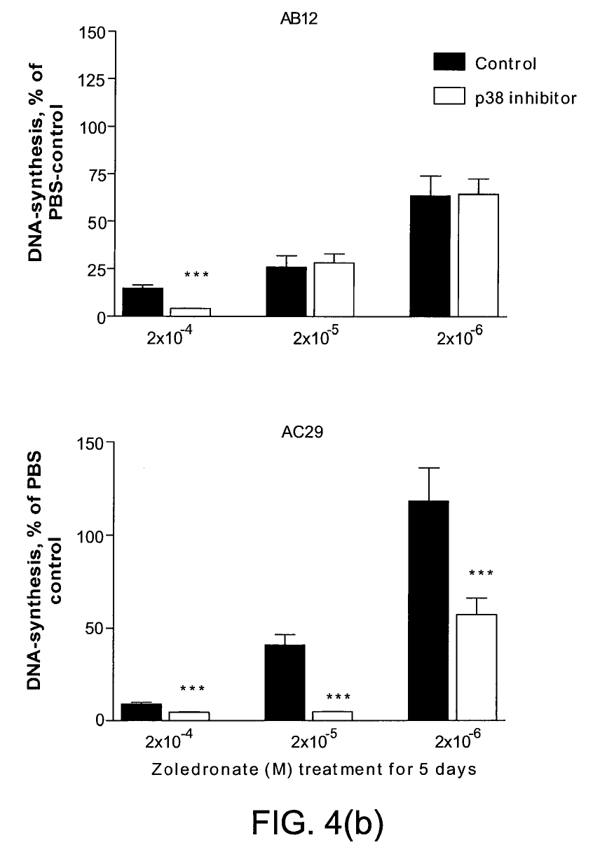

Inhibition of p38 augments n-BP induced growth inhibition. In addition to the inhibitory effects on the mevalonate pathway, n-BPs also activate the p38 MAP kinase in breast cancer cells. This activation signals for resistance against BP-induced growth inhibition, because blocking of the p38 MAPK pathway augments the growth inhibitory effects of BPs. A similar mechanism operates in mesothelioma cells. Using phospho-p38-specific and total p38 antibodies in Western blotting, risedronate and zoledronate were shown to induce a dose-dependent increase of p38 phosphorylation in AB12 and AC29 cells. Unlike accumulation of unprenylated Rap1A, this effect was not, however, reversible by excess (25 μM) geranylgeraniol. Increasing the geranylgeraniol dose did not affect the BP-induced, increased phosphorylation status of p38 either (FIG. 3). AB12 and AC29 cells were then cultured with risedronate or zoledronate, with or without the specific p38 inhibitor SB202190 (10⁻⁵ M) or with the same concentration of an inactive control compound SB202474. Inhibition of p38 augmented both risedronate- and zoledronate-induced growth inhibition in both cell lines, even though there were cell-specific differences between the BP-concentrations at which these effects were seen. In general, AC29 cells were more sensitive to the effects of p38 inhibition (FIG. 4).

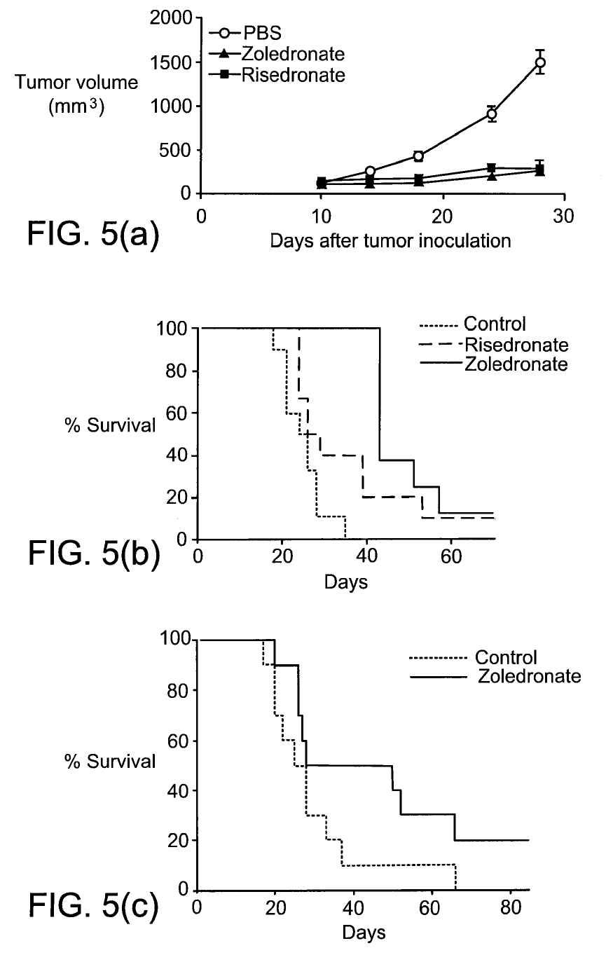

Risedronate and zoledronate inhibit mesothelioma growth in vivo. The antitumor activity of n-BPs was tested in vivo in a subcutaneous tumor model using AB12 cells, which are syngeneic in BALB/C mice, because AB12 tumors are more aggressive than the AC29 cells and have been resistant to most cancer chemotherapeutics in vivo. Groups of 10 BALB/c mice were inoculated s.c. with AB12 cells. Ten days later, when the tumors were palpable and between 100 to 175 mm³ in size, the mice were treated with risedronate or zoledronate, using higher doses and more infrequent, dosing schedules than previously applied in mouse tumor models. Inoculations with PBS served as a vehicle control Tumor volume was measured over time. Mice were sacrificed when tumors reached 2000 mm³. Both risedronate (P<0.02) and zoledronate (P<0.003) inhibited subcutaneous tumor growth (FIG. 5(a)). Neither of these BPs-treated tumors, however, completely regressed. The effects of risedronate and zoledronate on survival were examined in vivo in an intraperitoneal tumor model. Especially AB12 cells form diffuse tumors throughout the peritoneal cavity following i.p. injection, a pattern similar to the presentation of human peritoneal mesothelioma. Six days following i.p. inoculation of AB12 or AC29 cells, groups of 10-12 mice were treated by i.p. injection of either risedronate, zoledronate or PBS. Administration of zoledronate led to a significant increase in median survival (43 days for zoledronate versus 26 days for PBS; P<0.001). Median survival in the risedronate-group was 30 days. All PBS-treated mice died by day 35. In contrast, there were three long-term (> 60 days) and two long-term (>85 days) survivors in the risedronate and zoledronate-treatment groups, respectively (FIG. 5(b)), A similar survival experiment was also performed with mice bearing AC29 cells. After a total of 6 inoculations with the drug, the median survival of mice in the zoledronate-treatment group was 39 days, whereas, in the control group, the median survival was 26.5 days (FIG. 5(c)).

### Example 2

### Materials and Methods

Bisphosphonates. Stock solutions (10⁻² or 10⁻³ M) of nitrogen-containing bisphosphonates (risedronate, alendronate, pamidronate, and zoledronate) were prepared in PBS, pH was adjusted to 7.4 with NaOH and the solutions were filter-sterilized. Similarly, stock solutions of the non-nitrogen containing bisphosphonates (pyrophosphate-resembling bisphosphonates) clodronate (R₁=—Cl and R₂=—Cl) and etidronate (R₁=—OH and R₂=—CH₃) were prepared in PBS, pH was adjusted to 7.4 with NaOH and the solutions were filter-sterilized.

Cell culture. Human MDA-MB-231 breast cancer and mouse AB-12 mesothelioma cells were maintained in Dulbecco's modified Eagle's medium (Life Technologies, Inc.) supplemented with 10% fetal calf serum (HyClone Laboratories, Logan, Utah), 1% penicillin/streptomycin and non-essential amino acids (GIBCO BRL, Gaithersburg, Md.). All cell cultures were done in incubators in a 37° C. atmosphere of 5% CO₂/95% air.

Western blot analysis. Cells were cultured on 6-well plates in normal culture medium until near confluency. The cells were then rinsed with sterile PBS and cultured for further 24 h in serum-tree culture medium, in the presence or absence of the indicated bisphosphonates. Some cultures were also treated with 25 μM geranylgeraniol (cold, all trans), 1.0 μg/ml LPS (Sigma; St. Louis, Mo.) 10 ng/ml IL-1β (R&D Systems; Minneapolis, Minn.) or TNF-α (R&D Systems). To study bisphosphonate effects on connexin-43 expression, the cells were cultured for 24 h in serum-free culture medium in the presence of the indicated bisphosphonates or PBS as a vehicle control. Culture medium was then discarded, the cells were quickly lysed and prepared into Western blot samples, as described in Merrell et al. (2000) Breast Cancer Res. Treat. 81, 231-241. After boiling the supernatants in reducing sodium docedyl sulfate (SDS) sample buffer, equal amounts of protein (˜20-50 μg) were loaded per lane and the samples were electrophoresed on 10% SDS polyacrylamide gel and transferred to a nitrocellulose membrane. To detect, unprenylated Rap1A, the blots were incubated overnight at 4° C. with the antibody SC-1482 (Santa Cruz Technology (San Diego, Calif.)), diluted 1:1000 in Tris-buffered saline, 0.1% (v/v) Tween-20 (TEST), and then with peroxidase-conjugated anti-goat serum (Pierce; Rockford, Ill.), diluted 1:1000 in TBST. Total Rap1 was detected from stripped blots in a similar fashion, with the antibody SC-65 (Santa Cruz Technology). The phosphorylation status of p38 was investigated using anti-phospho-p38 (Cell Signaling; Beverly, Mass.) and after stripping of the membrane, with anti-total p38 antibodies (Cell Signaling), according to the manufacturer's instructions. Expression of connexin-43 was detected with a rabbit anti-connexin-43 antibody (Zymed Laboratories, Inc.; San Francisco, Calif.), diluted 1:1000 in TBST. The protein bands were visualized by chemiluminescence using SuperSignal West Pico ECL kit (Pierce Biotechnology, Inc.; Rockford, Ill.).

Cell viability assays. Cells were plated on 96-well plates at the density of 1×10³ cells in 100 μl per well in normal culture medium, with or without of the indicated concentrations of the various bisphosphonate combinations and or 1 mM CaCl₂, 1 mM EGTA or vehicle and cultured for 48 h. Cell viability was assessed with 3-(4,5-dimethylthiazol-2-yl)-5-(3-carboxy tetrazolium, inner salt (MTS)-assays (CellTiter Aqueous One 96 from Promega; Madison, Wis.) according to the manufacturer's instructions. For each treatment, a PBS-control group was run simultaneously on the same plate. The results for each treatment were calculated as % of the corresponding PBS-group.

Connexin-43 immunofluorescence staining. MDA-MB-231 cells were fixed with 3% paraformaldehyde in PBS, permeabilized with 1% TritonX-100 in PBS, blocked with 2% bovine serum albumin (BSA)-PBS, and then stained with the anti-connexin-43 antibody (diluted 1:50 in 2% BSA-PBS) and with the appropriate secondary antibody. The stainings were visualized using a Zeiss fluorescent microscope. Omission of the primary antibody served as a negative control for the staining.

Lucifer yellow uptake. To investigate the possibility that bisphosphonate-induced hemichannel opening mediates the effects of these drugs also in breast cancer cells Lucifer yellow uptake was studied in MDA-MB-231 cells. To avoid cell to cell dye transfer, the cells were seeded at the density of 30 000 cells/well, which resulted in a sparse distribution of the cells. The cells were first incubated in serum-free conditions with ethanol or heptanol for 15 minutes. After this, the treatments (vehicle, 5 mM EGTA, 10⁻⁶ M alendronate or zoledronate) were added for further 15 minutes. Lucifer yellow (10 μg/ml) (Sigma) was added for the final 1 minute after which the cells were washed with serum-free media to close the hemichannels again and fixed with 3% PFA-PBS. The cells were then stained with Hoechst (Sigma, 1 mg-ml stock prepared in ethanol and used in 1:800 dilution in PBS) to visualize nuclei, as previously described in Selander et al. (1996) Mol. Pharmacol. 50, 1127-1138. Samples were viewed with fluorescent microscope to examine the uptake of Lucifer yellow.

Flow cytometric analysis for γδTCR. MDA-231 cells were stained with a phycoerythrin-conjugated anti-γδ T-cell receptor (anti-γδ TCR) monoclonal antibody (clone 11F2; Becton Dickinson Biosciences; San Jose, Calif.). A phycoerythrin-conjugated, isotype matched irrelevant antibody served as a negative control. Cultured human blood mononuclear cells were used to exhibit positive staining. Analyses were performed using a FACSCalibur Flow cytometer (Becton Dickinson Biosciences; Franklin Lakes, N.J.) 7-aminoactinomycin D (Molecular Probes; Eugene, Oreg.) was used to exclude nonviable cells. Data analysis was performed using CellQuest software (Becton Dickinson Biosciences).

Tumor uptake of Tc99m-medronate. To investigate accumulation of the bone scanning agent (Tc99m-medronate) into the subcutaneous tumors, nude mice were first inoculated subcutaneously with AB-12 or MDA-MB-231 cells (10⁶ cells in 100 μl of sterile PBS) and the tumors were allowed to form for 3-4 weeks. Tc99m-medronate (MDP-Bracco™; Bracco Diagnostics Inc.; Princeton, N.J.) was then injected into the tail veins of the mice (˜800-1000 μCi per mouse) in 100 μl. High-resolution pinhole SPECT/CT imaging studies (X-SPECT system, GammaMedica, Inc.; Northridge, Calif.) and biodistribution analyses were performed in nude mice with xenografted rumors to measure in vivo tumor retention of Tc99 medronate following i.v. injection. For SPECT imaging, a total of 64 projections were acquired with a 30-sec acquisition time per projection, using a pinhole collimator with a 1-mm tungsten pinhole insert. Images were reconstructed using an ordered subsets expectation maximization (OSEM) algorithm with 20 iterations. In the CT system, the X-ray tube was operated at a voltage of 50 kV and an anode current of 0.6 mA. 256 projections were acquired to obtain the CT images, and acquisition time per projection was 0.5 second. The reconstructed images are 3 orientations with 1 mm mouse slices from the CT, SPECT, and fused SPECT/CT. The mice were terminated after imaging and tissues were collected and weighed ˜8 h after injection with the Tc99m-medronate. The dose retention, or % Injected Dose per gram, (% ID/g) of each tissue was calculated by measuring the radioactivity in the tissue using a gamma counter, decay correcting the count rate data to the Tc99m-medronate injection time and normalizing to the total injected dose in the animal as well as the tissue weight. Each animal's dose was determined by measuring the dosing syringe (AtomLab 100 dose calibrator; Biodex Medical Systems; Shirley, N.Y.) before and after injecting the mouse. To compare the accumulation of % ID/g between the breast cancer and mesothelioma-bearing mice, the % ID/g of the target tissue (subcutaneous rumor, femoral bone, heart muscle) was normalized against the % ID/g blood for each mouse.

Von Kossa staining to detect calcium deposits in mesothelioma and breast cancer tumors. To detect calcium minerals in the subcutaneously formed AB-12 and MDA-MB-231 tumors, they were first fixed in 1.0% neutral buffered formalin for 24 h and prepared into routine paraffin blocks. Sections of 5 μm in thickness were cut with a microtome. The sections were deparaffinized and hydrated through descending series of alcohol. The sections were placed in 5% silver nitrate solution and exposed to sunlight for 45 minutes, followed by 3 changes in deionized water. The sections were then treated in 2.5% sodium thiosulphate for 1 minute, rinsed well in deionized water, dipped for 3.5 seconds in 0.5% gold chloride and rinsed well in deionized water. The sections were counterstained with Van Gieson's Picro-fuchsin for 5 minutes, and finally dehydrated, cleared and mounted. Chemically cleaned and well rinsed glassware was used throughout the staining procedure. With this staining, calcium deposits are seen as dark brown to black precipitations.

Statistical analysis. All results are expressed as the mean±S.D., unless otherwise stated. Data were analyzed by Student's t-test. P values of < 0.05 were considered significant.

Results

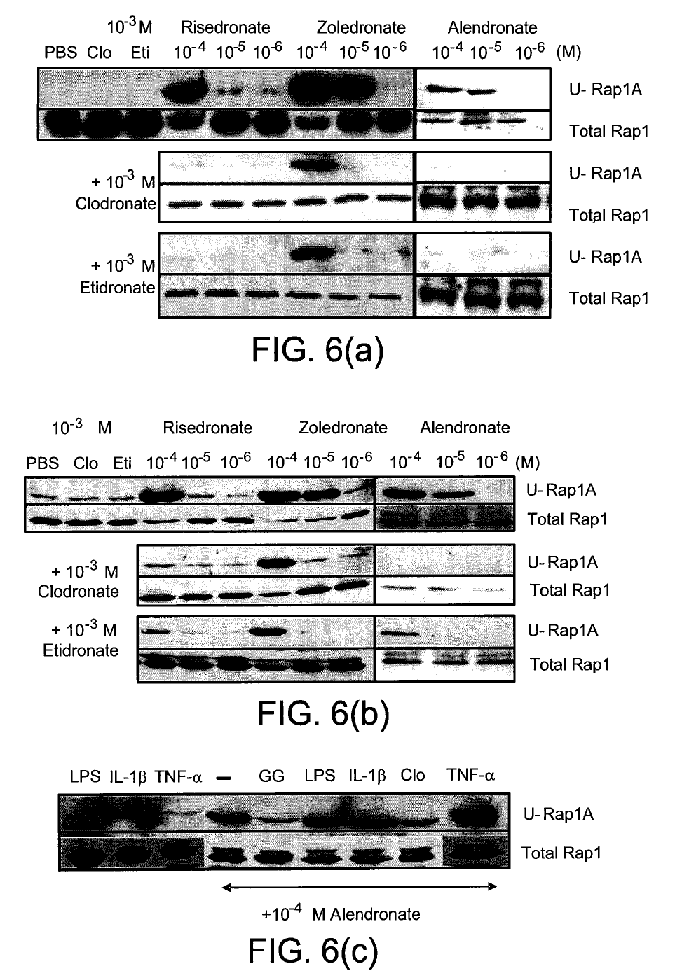

To investigate whether excess pyrophosphate-resembling bisphosphonates antagonize nitrogen-containing bisphosphonate-induced accumulation of unprenylated Rap1A and if a similar phenomenon can also be seen in cancer cells, MDA-MB-231 breast cancer and AB-12 mesothelioma cells were cultured with various concentrations of nitrogen-containing bisphosphonates (risedronate, zoledronate or alendronate) in the presence or absence of 10-1000 fold excess clodronate or etidronate for 24 h. The prenylation status of Rap1A in the cell lysates was studied with Western blots. As expected, clodronate or etidronate had no effect, but all the studied nitrogen-containing bisphosphonates induced a dose-dependent accumulation of unprenylated Rap1A, Zoledronate was the strongest inducer of this effect in both cell lines. In AB-12 cells the nitrogen-containing bisphosphonate-induced accumulation of unprenylated Rap1A was completely or partially reversed with both studied pyrophosphate-resembling bisphosphonates, depending on the concentration and the actual nitrogen-containing bisphosphonate that they were competed against (FIG. 6(a)). For example, when the pyrophosphate-resembling bisphosphonate: nitrogen-containing bisphosphonate ratio was 100:1, clodronate and etidronate completely blocked accumulation of unprenylated Rap1A induced by all nitrogen-containing bisphosphonates. Similar results were seen with MDA-MB-231 cells (FIG. 6(b)), For comparison, in addition to excess clodronate, alendronate-induced accumulation of unprenylated Rap1A in MDA-MB-231 cells was reduced only with the addition of excess geranylgeraniol (25 μM), but it was not blocked with TNF-α, IL-1β or LPS which are completely unrelated molecules to bisphosphonates (FIG. 6(c)).

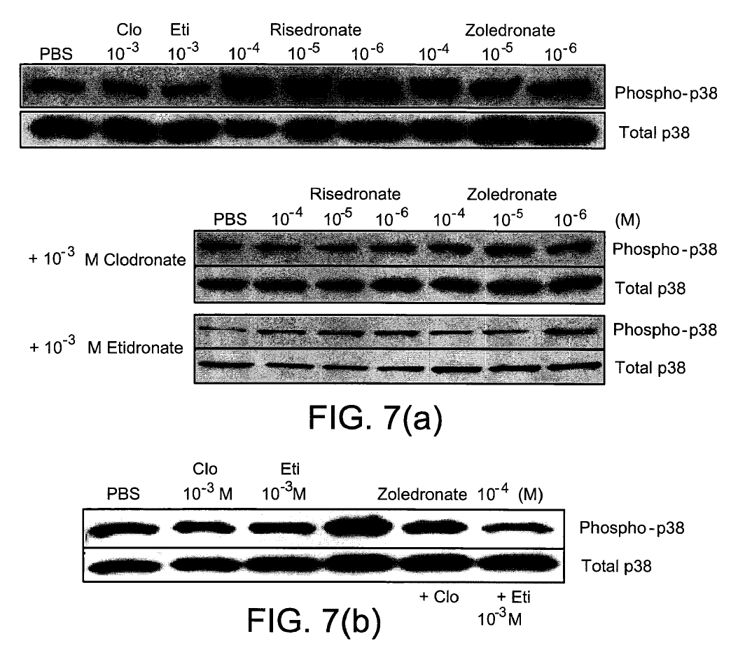

To determine whether excess pyrophosphate-resembling bisphosphonates antagonize nitrogen-containing bisphosphonate-induced phosphorylation of p38, the combined effects of nitrogen-containing bisphosphonates and pyrophosphate-resembling bisphosphonates on p38 activation were studied. Unlike earlier results with lower (10⁻⁵ M) concentrations, the high (10⁻³ M) concentrations of pyrophosphate-resembling bisphosphonates used here did not induce phosphorylation of p38 in either studied cell line. All studied nitrogen-containing bisphosphonates (10⁻⁴-10⁻⁶ M) did, however, induce p38 phosphorylation in AB-12 cells. The addition of excess clodronate or etidronate simultaneously with the nitrogen-containing bisphosphonates (risedronate or zoledronate) blocked this effect (FIG. 7(a)). Similar results also were seen in MDA-MB-231 cells where zoledronate-induced (10⁻⁴ M) p38 phosphorylation was blocked with both clodronate and etidronate (10⁻³ M) (FIG. 7(b)).

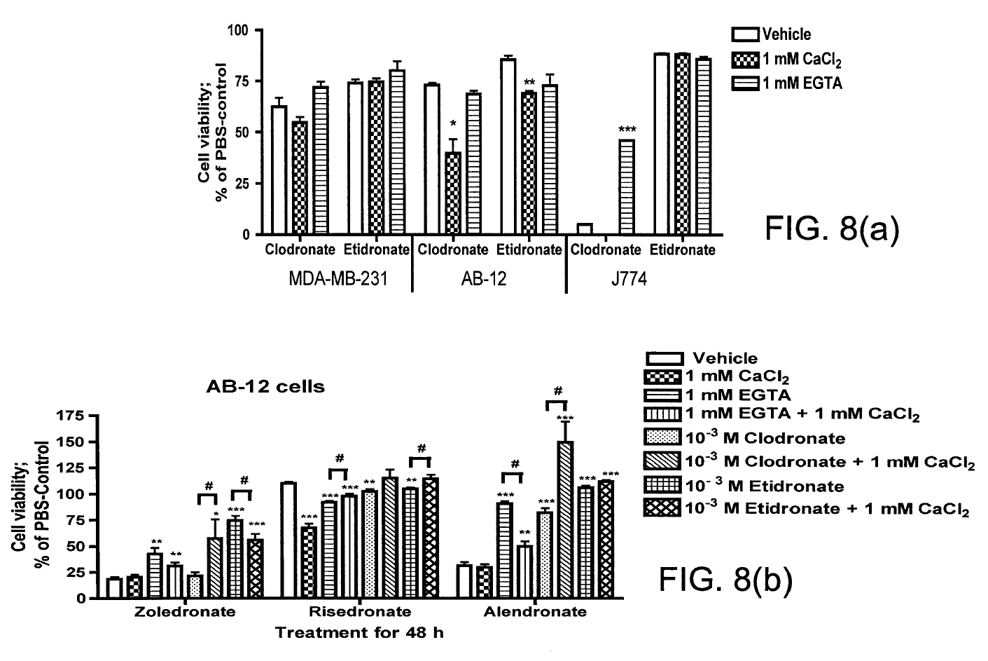

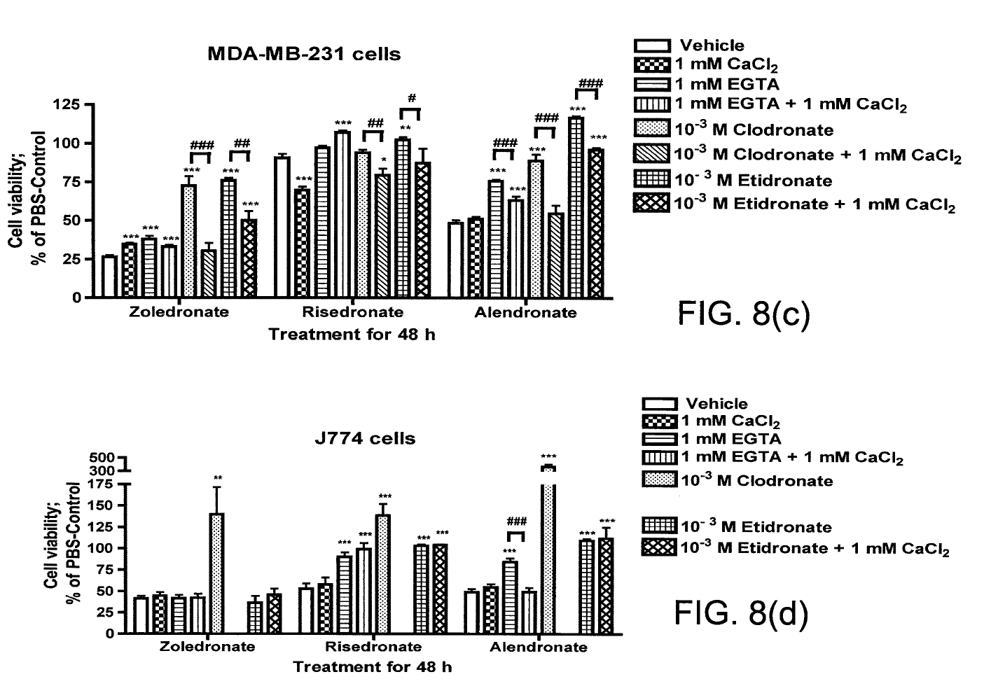

Next, whether excess pyrophosphate-resembling bisphosphonates antagonize the growth inhibitory effects of nitrogen-containing bisphosphonates was studied. First, the effects of the high pyrophosphate-resembling bisphosphonate doses (10⁻³ M) alone or in combination with 1 mM EGTA or Ca²⁺ on the viability of MDA-MB-23, AB-12 or J774 cells were assessed. The high doses of pyrophosphate-resembling bisphosphonates alone decreased the viability of all studied cells (P<0.001 vs. corresponding PBS-control). The J774 macrophage-like cells exhibited the highest sensitivity to the growth inhibitory effects of clodronate, which was reversed by addition of 1 mM EGTA. Addition of 1 mM CaCl₂ did not affect the growth-inhibitory effects of pyrophosphate-resembling bisphosphonates in MDA-MB-231 cells, but enhanced those in AB-12 cells. The combination of clodronate and 1 mM CaCl₂ was toxic to J774 cells. Otherwise, addition of EGTA or CaCl₂ did not interfere with pyrophosphate-resembling bisphosphonate effects on viability in these cell lines (FIG. 8(a)).

Next whether excess (10⁻³ M) pyrophosphate-resembling bisphosphonates (clodronate or etidronate) affect the cell viability changes induced by 10⁻⁴ M nitrogen-containing bisphosphonates (alendronate, risedronate, or zoledronate) was examined. All nitrogen-containing bisphosphonates, except for risedronate, induced a significant decrease in cell viability (P<0.001) in MDA-MB-231 and AB-12 cells. In J774 cells, also risedronate significantly decreased cellular viability. The obvious growth-inhibitory effects of the nitrogen-containing bisphosphonates were reversed by pyrophosphate-resembling bisphosphonates. There were, however, cell- and drug-specific exceptions to these results. The growth inhibitory effects of zoledronate were not reversed by clodronate in AB-12 and by etidronate in J774 cells. There were also differences in cellular responses to the combination of risedronate and pyrophosphate-resembling bisphosphonates; Clodronate decreased slightly, but significantly cell viability when these two drugs were given simultaneously to AB-12 cells, but it did not interfere with risedronate effects in MDA-MB-231 cells. In J774 cells, clodronate significantly reversed the risedronate-induced decrease in viability. When compared with vehicle+risedronate-treatment, etidronate+risedronate-treatment decreased cell viability in AB-12 cells but increased it in MDA-MB-231 cells. In J774 cells, etidronate reversed risedronate-induced decrease in viability (FIGS. 8(b)-8(d)).

Additionally, whether manipulating culture medium Ca²⁺ concentrations affects the ability of pyrophosphate-resembling bisphosphonates to antagonize the effects of nitrogen-containing bisphosphonates on cellular viability was investigated. The results again were bisphosphonate- and cell-specific. Addition of Ca²⁺ slightly reversed the growth inhibitory effects of zoledronate in MDA-MB-231 cells and enhanced tire growth inhibitory effects of risedronate in both cancer cell lines. Surprisingly, in J774 cells, excess Ca²⁺ did not augment the nitrogen-containing bisphosphonate effects on viability. Addition of EGTA reversed zoledronate- and alendronate-induced growth inhibition in both cancer cell lines and enhanced the growth inhibitory effects of risedronate in AB-12 cells. In J774 cells, EGTA reversed the growth inhibitory effects of risedronate and alendronate. Excess Ca²⁺ significantly decreased the EGTA effect in reversing alendronate-induced growth inhibition of all three cell lines. Although the same was seen in the zoledronate-group in the cancer cell lines, the effects were not statistically significant Excess Ca²⁺ also reversed the ability of EGTA to potentiate risedronate-induced growth inhibitory effects in AB-12 cells. In MDA-MB-231 cells, simultaneous addition of Ca²⁺ with EGTA increased viability in the risedronate-group, as compared with the corresponding risedronate+vehicle-treated control. Addition of 1 mM CaCl₂ simultaneously with clodronate or etidronate decreased the ability of these pyrophosphate-resembling bisphosphonates to protect against nitrogen-containing bisphosphonate-induced decrease in viability in MDA-MB-231 cells. The results were the opposite with clodronate and Ca²⁺ in AB-12 cells, where Ca²⁺ potentiated the protective effects of clodronate against zoledronate and alendronate. Similar effects were also seen with etidronate and Ca²⁺ in the risedronate-group in AB-12 cells. Addition of excess Ca²⁺, however, either significantly decreased or did not interfere with the protective effect of etidronate against zoledronate or alendronate, respectively, in the AB-12 cells. In J774 cells, etidronate effects against nitrogen-containing bisphosphonates were not affected by Ca²⁺ and the presence of clodronate with Ca²⁺ was toxic in all treatment groups (FIGS. 8(b)-8(d)).

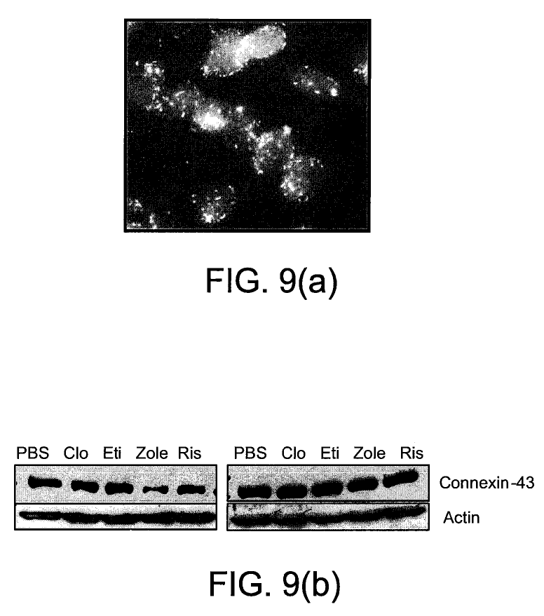

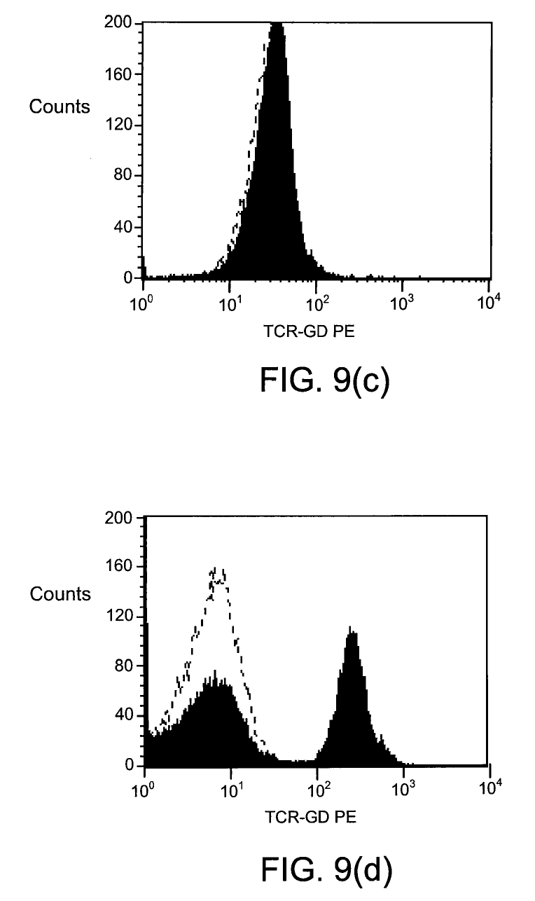

Next, to determine whether treatment with nitrogen-containing bisphosphonates does not increase hemichannel mediated uptake in MDA-MB-231 cells the expression of connexin-43 hemichannel and γλTCR proteins in MDA-MB-231 cells was analyzed, γλTCR expression was detected with flow cytometry in peripheral blood monocytes, but not in MDA-MB-231 cells. Connexin-43 expression was seen on the cell membranes of MDA-MB-231 cells using immunofluorescence. Treatment for 24 h with zoledronate (10⁻⁴ M), but not with any other tested bisphosphonate, slightly decreased the connexin-43 expression (FIG. 9). Treatment of the MDA-MB-231 cells with EGTA increased the uptake of Luciferin yellow from the surrounding culture medium, and this was preventable with heptanol, suggesting that the connexin-43 mediated uptake is functional in these cells. Nitrogen-containing bisphosphonates did not, however, increase the uptake of Luciferin yellow in these cells.

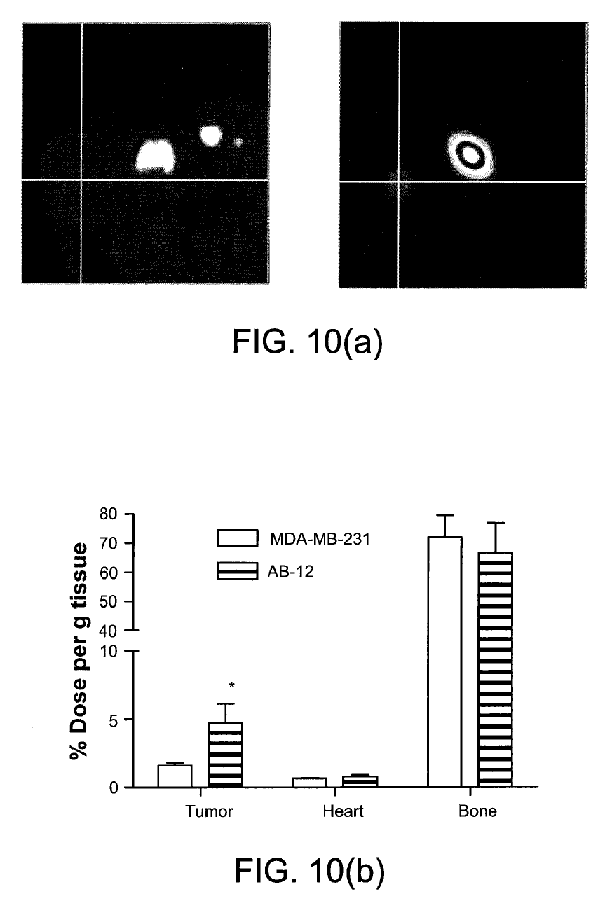

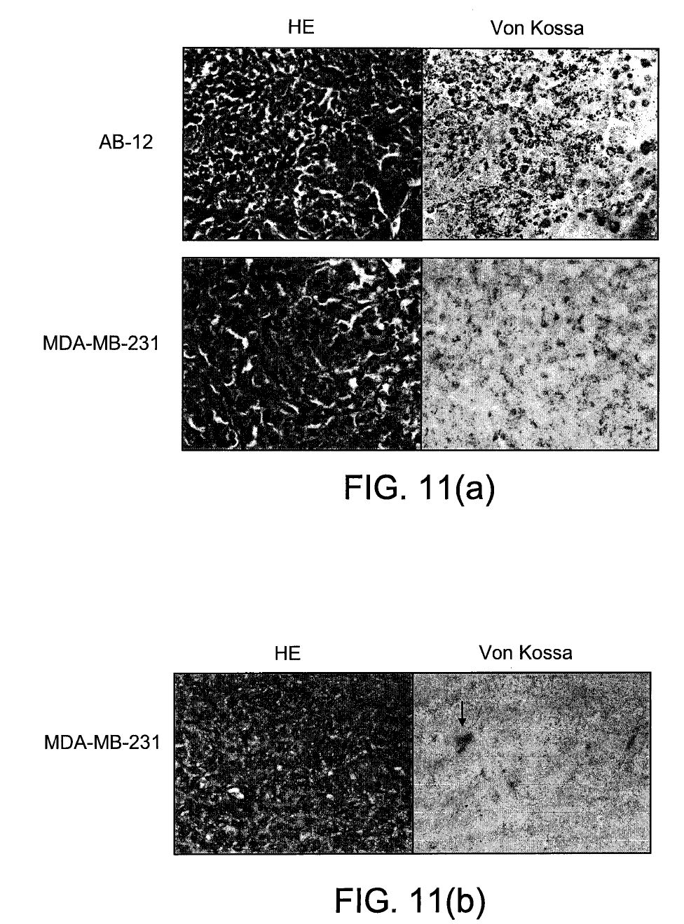

To investigate bisphosphonate-uptake into tumors in vivo, i.e., whether mesothelioma tumors exhibit higher Tc99m-medronate uptake than breast cancer tumors, MDA-MB-231 and AB-12 cells were inoculated subcutaneously into nude mice and tumors were allowed to form. The animals were then injected with the bone scanning agent Tc99m-medronate and the % dose retention was analyzed in various tissues. The highest proportion of the drug accumulated in the bones. Furthermore, accumulation of radioactivity was similar in the hearts and femoral bones in both groups of mice that were bearing either breast cancer or mesothelioma tumors. Accumulation of Tc99m-medronate was, however, significantly higher in the mesothelioma tumors formed by the AB-12 cells, as compared with breast cancer tumors formed by the MDA-MB-231 cells (FIG. 10). Finally, to investigate the mechanisms through which the bone scanning agent is retained within the tumors, the tumors were analyzed via Von Kossa-stainings, which detect Ca²⁺-minerals. In both tumor types, patchy, intracellular positive staining for Ca²⁺-minerals was detected. In the mesothelioma tumors, staining was only seen in areas of tumor necrosis, in breast cancer tumors, cells surrounding necrotic areas stained positive with Von Kossa. No positive staining was seen in areas of viable tumors formed by AB-12 cells and only rarely in individual cells of viable tumors formed by MDA-MB-231 cells (FIG. 11).

in this example, the effects of bisphosphonates in mesothelioma and breast cancer cells, which have been shown, to exhibit different sensitivities to the growth-inhibitory effects of bisphosphonates in vivo, were compared. The data show that accumulation of Tc99m-medronate, a bisphosphonate that is clinically used in bone scans, is significantly higher in subcutaneous mesothelioma tumors, as compared with subcutaneous breast tumors. Although accumulation of medronate cannot be considered to represent the accumulation of all bisphosphonates into tumors at the soft tissue sites, the results suggest that the increased sensitivity of mesothelioma cells to the growth-inhibitory effects of bisphosphonates in vivo, may be related to their increased intratumoral accumulation of these drugs.

These data further demonstrate that pyrophosphate-containing bisphosphonates block nitrogen-containing bisphosphonate-induced effects also in breast cancer and mesothelioma cells. Further, the effects of bisphosphonates on cellular viability can be regulated by affecting the culture medium Ca²⁺-concentration. There are, however, significant cell and drug-specific differences in how cells respond to the combination of bisphosphonates and Ca²⁺. For example, the data show that excess Ca²⁺ reverses the ability of zoledronate to decrease viability in MDA-MB-231 breast cancer cells, but not in AB-12 mesothelioma cells. Also, addition of Ca²⁺ augmented the growth inhibitory effects of clodronate and etidronate in AB-12 cells, but not in MDA-MB-231 cells. Without being bound by theory, these results show that the antagonistic effects of excess pyrophosphate-resembling bisphosphonates against nitrogen-containing bisphosphonates may be explained by their ability to chelate calcium, resulting in decreased cellular up-take of nitrogen-containing bisphosphonates, which is Ca²⁺ dependent. Therefore, there may be a step or steps during the intracellular processing of bisphosphonates for which the various drug molecules compete. Changes in J774 viability when the cells were cultured with excess Ca²⁺ and nitrogen-containing bisphosphonates were not observed, as compared with treatment with nitrogen-containing bisphosphonates alone. The only situation where excess Ca²⁺ augmented the nitrogen-containing bisphosphonate-induced decrease in cellular viability was seen with risedronate. Taken together, the results show that the combination of extracellular Ca²⁺ and bisphosphonates have cell and drag molecule specific effects on cell viability.

MDA-MB-231 breast cancer cells express connexin-43 on their cell membranes and treatment with a high dose of zoledronate for 24 h appeared to slightly decrease the expression of connexin-43, but increased hemichannel-mediated cellular uptake of Lucifer yellow in response to short-term alendronate or zoledronate-treatment was not detected. These results show that the nitrogen-containing bisphosphonate effects on hemichannels are cell-specific and do not necessarily occur in breast cancer cells. γλT-cell receptor expression was not detected in these breast cancer cells either; thus nitrogen-containing bisphosphonates do not affect MDA-MB-231 breast cancer cells via this receptor.

These data further indicate that mesothelioma cells have a higher capacity to accumulate bisphosphonate in vivo. The cellular effects of nitrogen-containing bisphosphonates can be overcome by excess pyrophosphate-resembling bisphosphonates in both mesothelioma and breast cancer cells. Without being bound by theory, this can be explained in part by Ca²⁺-chelation by the pyrophosphate-resembling bisphosphonates, and thereby, decreased cellular up-take of the nitrogen-containing bisphosphonates. There could be additional steps in the intracellular processing of these drugs for which the different molecules compete. The results further show that the cancer growth inhibiting effects of bisphosphonates may be affected by extracellular Ca²⁺ in a cancer cell- and bisphosphonate-specific fashion. Since calcifications are frequently seen in malignant tumors, tumor calcification would affect the outcomes of bisphosphonate-treatment in tumors that are growing at visceral sites.

The patents and publications mentioned herein are incorporated by reference herein in their entirety to the same extent as if each individual publication was specifically and individually indicated to be incorporated by reference.

The present methods and compositions are not limited in scope by the embodiments disclosed in the examples which are intended as illustrations of a few aspects of the methods and compositions and any embodiments which are functionally equivalent are within the scope of the claims. Various modifications of the methods and kits in addition to those shown and described herein will become apparent to those skilled in the art and are intended to fall within the scope of the appended claims. Further, while only certain representative combinations of the compositions disclosed herein are specifically discussed in the embodiments above, other combinations of the compositions will become apparent to those skilled in the art and also are intended to fall within the scope of the appended claims. Thus a combination of steps or compositions may be explicitly mentioned herein; however, other combinations of steps or compositions are included, even though not explicitly stated.

1. A method of treating a subject with mesothelioma or at risk of developing mesothelioma comprising administering to the subject a nitrogen-containing bisphosphonate.

2. The method of claim 1, wherein the nitrogen-containing bisphosphonate is selected from the group consisting of alendronate, ibandronate, minodronate, neridronate olpadronate, pamidronate, risedronate, and zoledronate.

3. The method of claim 1, wherein the nitrogen-containing bisphosphonate is alendronate.

4. The method of claim 3, wherein the alendronate is administered at a dose of about 0.1 mg/day to about 100 mg/day.

5. The method of claim 3, wherein the alendronate is administered at a dose of up to about 70 mg/day.

6. The method of claim 1, wherein the nitrogen-containing bisphosphonate is pamidronate.

7. The method of claim 6, wherein the pamidronate is administered at a dose of about 0.1 mg/day to about 120 mg/day.

8. The method of claim 6, wherein the pamidronate is administered at a dose of up to about 90 mg/day.

9. The method of claim 1, wherein the nitrogen-containing bisphosphonate is risedronate.

10. The method of claim 9, wherein the risedronate is administered at a dose of about 0.1 mg/day to about 50 mg/day.

11. The method of claim 9, wherein the risedronate is administered at a dose of up to about 30 mg/day.

12. The method of claim 1, wherein the nitrogen-containing bisphosphonate is zoledronate.

13. The method of claim 12, wherein the zoledronate is administered at a dose of about 0.1 mg/day to about 5 mg/day.

14. The method of claim 12, wherein the zoledronate is administered at a dose of up to about 4 mg/day.

15. The method of claim 1, wherein the nitrogen-containing bisphosphonate is administered once per day.

16. The method of claim 1, wherein the nitrogen-containing bisphosphonate is administered in multiple doses.

17. The method of claim 1, further comprising administering to the subject a p38 inhibitor.

18. The method of claim 17, wherein the p38 inhibitor is SB202190.

19. The method of claim 17, wherein the p38 inhibitor is administered at the same time as the nitrogen-containing bisphosphonate.

20. The method of claim 1, further comprising identifying a subject with or at risk of developing mesothelioma prior to the administration step.

21. A composition comprising a nitrogen-containing bisphosphonate and a p38 inhibitor.

22. The composition of claim 21, wherein the nitrogen-containing bisphosphonate is selected from the group consisting of alendronate, ibandronate, minodronate, neridronate, olpadronate, pamidronate, risedronate, and zoledronate.

23. The composition of claim 21, wherein the nitrogen-containing bisphosphonate is alendronate.

24. The composition of claim 21, wherein the nitrogen-containing bisphosphonate is pamidronate.

25. The composition of claim 21, wherein the nitrogen-containing bisphosphonate is risedronate.

26. The composition of claim 21, wherein the nitrogen-containing bisphosphonate is zoledronate.

27. The composition of claim 21, wherein the p38 inhibitor is SB202190.

28. A kit comprising a composition comprising a nitrogen-containing bisphosphonate and instructions for administering the composition to a subject with mesothelioma or at risk of developing mesothelioma.

29. The kit of claim 28, wherein the nitrogen-containing bisphosphonate is selected from the group consisting of alendronate, ibandronate, minodronate, neridronate, olpadronate, pamidronate, risedronate, and zoledronate.

30. The kit of claim 28, further comprising a p38 inhibitor.

31. The kit of claim 29, wherein the p38 inhibitor is SB202190.
